(19) United States
(12) Patent Application Publication
(10) Pub. No.: US 2021/0085994 A1
(43) Pub. Date: Mar. 25, 2021

(54) SYSTEMS AND METHODS FOR MODULATING CYTOKINE ACTIVITY

(71) Applicant: The Trustees of Columbia University in the City of New York, New York, NY (US)

(72) Inventor: Sinisa Vukelic, New York, NY (US)

(21) Appl. No.: 17/031,746

(22) Filed: Sep. 24, 2020

Related U.S. Application Data
(63) Continuation of application No. PCT/US2019/024321, filed on Mar. 27, 2019.
(60) Provisional application No. 62/648,851, filed on Mar. 27, 2018.
(60) Provisional application No. 62/654,008, filed on Apr. 6, 2018.

(51) Int. Cl. A61N 5/06

(52) U.S. Cl. CPC A61N 5/0613; A61N 2005/067; A61N 5/0601

(57) ABSTRACT

The disclosed subject matter provides systems and methods for modulating cytokine activity in a tissue. The system and methods can be used in a variety of tissues including the cornea. The methods of the disclosed subject matter includes a method of modulating cytokine activity within a cornea without treating keratoconus or altering curvature of the cornea, the method comprising: controlling a light source to apply light energy pulses to corneal tissue; wherein the light energy pulses: (a) are below an optical breakdown threshold for the cornea; (b) ionize water molecules within the treated corneal layer to generate reactive oxygen species; and modulate cytokine activity.

### CROSS-REFERENCE TO RELATED APPLICATIONS

This application is a continuation of International Application No. PCT/US2019/024321, filed Mar. 27, 2019, which claims the benefit of priority to U.S. Provisional Patent Application Ser. Nos. 62/648,851, filed Mar. 27, 2018, and 62/654,008, filed Apr. 6, 2018. The entire content of each of these applications is hereby incorporated by reference herein.

### BACKGROUND

Cytokines are small proteins that are important in signaling in between cells, which is an important function especially in tissue repair where signaling between different cell types is important.

### SUMMARY

Embodiments of the disclosed subject matter provide a method of modulating cytokine activity within a cornea without treating keratoconus or altering curvature of the cornea. The method includes: controlling a light source to apply light energy pulses to corneal tissue; wherein the light energy pulses: are below an optical breakdown threshold for the cornea; ionize water molecules within the treated corneal layer to generate reactive oxygen species; and modulate cytokine activity. In some embodiments, the light source is a laser. In some embodiments, the laser is a femtosecond laser.

In some embodiments, the light energy pulses have an average power output between about 10 mW and about 100 mW. In some embodiments, the light energy pulses have a pulse energy between about 0.1 nJ and about 1 μJ. In some embodiments, the light energy pulses have a wavelength between about 200 nm and about 1600 nm. In some embodiments, the light energy pulses have a wavelength that is not significantly absorbed by amino acids in collagen.

In some embodiments, the modulated cytokine activity includes one or more selected from the group consisting of: expression and signaling. In some embodiments, the expression comprises protein expression. In some embodiments, the signaling comprises inflammatory signaling. In some embodiments, the signaling modulates cytokine activity of one or more types of cells. In some embodiments, the one or more types of cells comprise keratocytes. In some embodiments, the cytokine is selected from the group consisting of: a chemokine, an interferon, an interleukin, a lymphokine, and a tumor necrosis factor. In some embodiments, the interleukin includes one or more selected from the group consisting of: interleukin 1 and interleukin 2.

In some embodiments, any the methods of the disclosed subject matter as described herein is performed for prophylactic purposes as part of a surgical corrective procedure. In some embodiments, the surgical corrective procedure is selected from the group consisting of: laser-assisted in situ keratomileusis (LASIK), photorefractive keratectomy (PRK), laser-assisted sub-epithelial keratectomy (LASEK), phakic intraocular lens implantation, radial keratotomy, and cataract surgery.

Embodiments of the disclosed subject matter provide a system for treating a cornea, the system comprising: a light source configured to project light energy pulses onto at least a portion of a cornea; and a controller programmed to control the light source in accordance with any of method as described herein. In some embodiments, the system further includes: an imaging device configured to image the cornea. In some embodiments, the imaging device is further configured to perform one or more techniques selected from the group consisting of: en face imaging, tomography, and topographic imaging.

Embodiments of the disclosed subject matter provide a system for adapting a laser system for treating a cornea, the system including: laser modification optics adapted and configured to adjust laser output of the laser system; and a controller programmed to control the laser modification optics as the light source in accordance with any of methods as described herein. In some embodiments, the system further includes: an imaging device configured to image the cornea. In some embodiments, the imaging device is further configured to perform one or more techniques selected from the group consisting of: en face imaging, tomography, and topographic imaging.

Embodiments of the disclosed subject matter provide a method of preventing or decreasing inflammation, scar formation, or cytokine activity in a non-ophthalmologic and non-arthroscopic procedure, the method including: controlling a light source to apply light energy pulses to non-ophthalmologic and non-cartilaginous tissue; wherein the light energy pulses: are below an optical breakdown threshold for the non-ophthalmologic and non-cartilaginous tissue; ionize water molecules within the treated non-ophthalmologic and non-cartilaginous tissue to generate reactive oxygen species that cross-link collagen within the non-ophthalmologic and non-cartilaginous tissue; and modulate cytokine activity.

In some embodiments, the non-ophthalmologic and non-cartilaginous tissue is selected from the group consisting of: skin, tendon, ligament, neural, vascular, muscle, and bone. In some embodiments, the light energy pulses are applied to one or more selected from the group consisting of: a wound and an implant pocket.

### BRIEF DESCRIPTION OF THE DRAWINGS

For a fuller understanding of the nature and desired objects of the disclosed subject matter, reference is made to the following detailed description taken in conjunction with the accompanying drawing figures wherein like reference characters denote corresponding parts throughout the several views.

FIG. 1 depicts a schematic of an exemplary method of the disclosed subject matter.

FIG. 2 depicts two-photon fluorescence (TPF) images of cross-sections of (a) control and laser-treated pig eyes. Three regions are imaged in the treated eye: an untreated region (left), a transitional region (middle) and the central region (right). The scale bar is 60 μm.

FIG. 3 depicts histological sections of hematoxylin-eosin (H&E)-stained rabbit corneas two days after treatment (Panel (a)); seven days after treatment (Panel (b)); and three months after treatment (Panel (c)). Panels (d), (e), and (f) depict the corresponding untreated controls. The scale bar is 100 μm.

FIGS. 4A-4B depicts in vivo images of a rabbit eye four days after epithelium debridement. FIG. 4A depicts an exemplary rabbit eye where the epithelium was removed and left to heal on its own. FIG. 4B depicts an exemplary rabbit eye where the epithelium was debrided and the eye was then subjected to the laser treatment. The de-epithelized, injured region (dyed area) is much small in the laser-treated, indicating that the laser irradiation enhances healing.

FIG. 5A depicts an experimental set-up. FIG. 5B depicts a schematic diagram depicting laser beam delivered to the eye ex vivo. The eye is placed in a custom-built eye chamber.

FIGS. 6A and 6B depict an exemplary experimental set-up for live animal treatment. Rabbits were deeply anesthetized and placed in a customized holder. FIG. 6C depicts the treatment, which involved applying laser pulses such that the laser followed a zigzag trajectory over a circle of diameter 5 mm, resulting in the treatment of a planar area at a specific depth. The treatment was performed at five different depths, effectively inducing ‘treatment layers’. Multiple treatment layers parallel to the surface were created, with a distance of 50 μm between consecutive planes.

FIGS. 7A-7E provide representative in vivo confocal microscopy images of laser-treated (rows I, III, V, VII and IX) and control (rows II, IV, VI, VIII and X) rabbit eyes obtained 48 hours (FIG. 7A; rows I and II), 7 days (FIG. 11B; rows III and IV), one month (FIG. 7C; rows V and VI), two months (FIG. 7D; rows VII and VIII) and three months (FIG. 7E; rows IX and X) after treatment. (a˜j)—corneal epithelium; (k˜t)—keratocyte network; (u˜z, aa-dd)—corneal endothelium (scale bar=50 μm).

FIGS. 8A-8B illustrate rabbit corneal keratocyte (FIG. 8A) and endothelium (FIG. 8B) cell densities in vivo, on days 2 and 7, one month, two months and three months after laser treatment.

FIGS. 9A-9B depict DSC thermograms of untreated (FIG. 9A) and laser-treated (FIG. 9B) samples of pig cornea. The thermal denaturation temperature of the treated samples is about 2° C. higher than that for untreated samples. Comparison of the averaged data for the control and treated samples confirms the higher denaturation temperature for the laser-irradiated samples (FIG. 9B).

FIGS. 10A-10C illustrate temperature measurement of femtosecond irradiated corneal tissue. FIG. 10A depicts an exemplary experimental setup and FIG. 10B depicts the lines along which the measurements were taken. FIG. 10C depicts temperature distribution as a function of distance from the focal volume.

FIGS. 11A-11B provide representative Nomarski images of control (FIG. 11A) and laser-irradiated (FIG. 11B) corneal tissue 24 hours after laser treatment. The treated region is boxed. The images demonstrate the absence of hazing after the laser treatment. The scale bar is 100 μm.

FIG. 12 depicts exemplary live/dead staining of corneal punch specimens. Panel (a) depicts control and Panel (b) depicts laser-treated specimens at 24 h. Panel (c) depicts control and Panel (d) depicts laser-treated specimens at 1 week. Live cells are labeled green and dead cells are red. The scale bar is 200 μm.

FIGS. 13A-13C depicts hematoxylin and eosin (H&E)-stained histological cross-sections of untreated control after 1 week of culture (FIG. 13A); femtosecond laser-irradiated pig corneas after 24 hour (FIG. 13B) and 1 week (FIG. 13C) of in situ incubation. Blue dots represent keratocytes. The scale bar is 100 μm.

FIG. 14 illustrates hematoxylin and eosin (H&E)-stained histological cross-sections of the anterior portion of femtosecond laser-irradiated pig corneas (left panel) and untreated controls (right panel), both after one week of in situ incubation. Blue dots represent keratocytes. The scale bar is 100 μm.

FIG. 15 depicts two-photon fluorescence (TPF) images of cross-sections of (a) control and (b) laser-treated pig eyes. Three regions are imaged in the treated eye: the untreated region (left), the transitional region (middle) and the central region (right), as in the procedure performed on eyes ex vivo (shown in the main body of the study). The control sample and the untreated region of the laser-irradiated specimen after one week in culture ex vivo had a similar crosslink density to the 24 hour sample in the ex vivo study. Samples cultured for one week had a larger laser-irradiated region, due to swelling of the corneal tissue during culture.

FIG. 16, comprising rows A, B and C, depicts laser scanning confocal microscopy (LSCM) images of stromal keratocyte. Row A depicts panels of control images, wherein no epithelium debriding and laser treatment were applied. Row B depicts panels of images wherein the epithelium was debrided, but no laser treatment was applied. Row C depicts panels of images wherein the epithelium was debrided, and the laser treatment was applied.

FIG. 17 depicts quantitative analysis of keratinocyte density (cells/mm³) in corneas of rabbit eyes at the 24-hour time point of the procedure. Group 1 is the control group; Group 2 includes samples wherein the epithelium was debrided but were not laser treated; Group 3 includes samples where the epithelium was debrided and laser treated.

FIG. 18 comprising rows A, B and C, depicts laser scanning confocal microscopy (LSCM) images of epithelial monolayers. Row A depicts panels of control images, where no epithelial debriding and laser treatment were applied. Row B depicts panels of images wherein the epithelium was debrided, but no laser treatment was applied. Row C depicts images where the epithelium was debrided, and the laser treatment was applied.

FIGS. 19A and 19B depict quantitative analysis of endothelial cell counts (cells/mm²) at the 24-hour time point. Group 1 includes control cells (no epithelial debridement and no laser treatment), Group 2 includes cells that were debrided but not laser treated, and Group 3 includes cells that were debrided and laser treated. FIG. 19A depicts mean values for each group. FIG. 19B depicts a summary of these results.

FIG. 20 depicts results from replicates of quantitative ELISA of serial dilutions of IL-1β solutions that were either laser-treated (treated, orange squares) or untreated (control, blue diamonds).

FIG. 21 depicts results from quantitative ELISA of serial dilutions of the laser-treated (diamonds) and untreated (squares) samples of IL1R1 solutions.

FIGS. 22A and 22B depict results from quantitative ELISA of IL-1β-IL1R1 interactions utilizing control (untreated) or laser-treated (treated) IL-1β solutions and IL1R1-coated microplates. FIG. 22A depicts results from plates treated with IL-1β solutions at concentrations of 1.2 μg/mL and 0.3 μg/mL. FIG. 22B depicts results from plates treated with IL-1β solutions at concentrations of 1 μg/mL, 0.5 μg/mL and 0.3 μg/mL.

### DEFINITIONS

The instant disclosed subject matter is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Embodiments of the disclosed subject matter provide methods, computer-readable media, and systems for modulating cytokine activity, e.g., within a cornea. Other embodiments of the disclosed subject matter provide methods, computer-readable media, and systems for preventing or decreasing inflammation, scar formation, or cytokine activity. Embodiments of the disclosed subject matter can be performed within the eye without treating keratoconus (e.g., by performing methods of the disclosed subject matter of a subject that is not in need of treatment for keratoconus) or altering curvature of the cornea (e.g., by performing methods of the disclosed subject matter of a subject that is not in need of vision correction or in association with an alternative vision correction procedure). Additionally, embodiments of the disclosed subject matter can be used in non-ophthalmologic and non-cartilaginous tissues and procedures.

Embodiments of the disclosed subject matter can be applied to a variety of tissue including cartilage, articular cartilage, and the like.

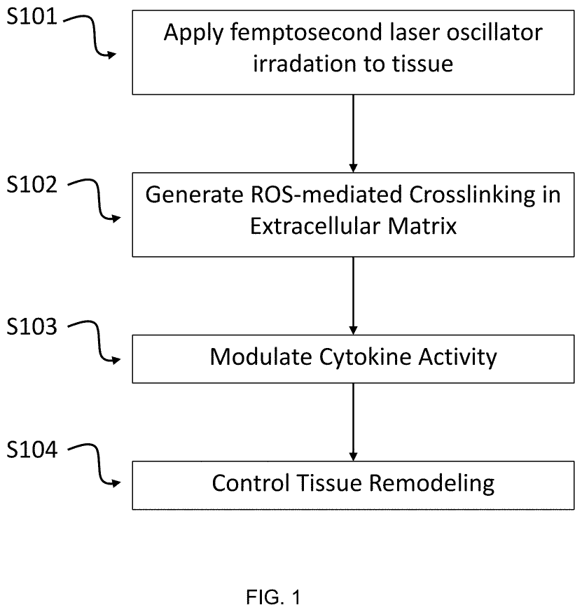

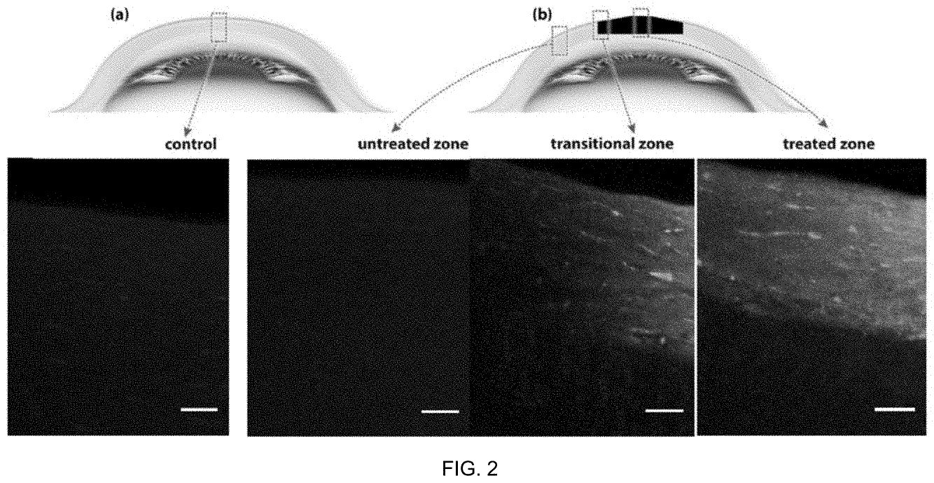

Referring now to FIG. 1, an example of a method of modulating cytokine activity within a tissue, for example a cornea is shown.

In step S101, a light source irradiates tissue. In some embodiments, the light source is a femtosecond laser oscillator. In some embodiments, the tissue is eye tissue, for example, tissue of the cornea including corneal flap tissue, stromal tissue, retinal tissue, and the like. In some embodiments, the tissue is non-ophthalmologic tissue. For example, in some embodiments the tissue is collagenous tissue, such as one or more ligaments, tendons, skin, bone, vascular tissue, fascia, cartilage including articular cartilage, or other connective tissue. In some embodiments, the skin is regions of skin where tissue remodeling has occurred or where tissue remodeling and/or scar reduction may be desired (e.g., regions including scarred skin, wrinkled skin, burned skin, and the like). In some embodiments, the tissue includes nerve tissue, bone tissue, muscle tissue, adipose tissue. In some embodiments, the tissue includes one or more epithelial tissues including simple epithelial tissue, stratified epithelial tissue and the like.

In step S102, the femtosecond laser induces a low-density plasma that generates an ionization field resulting in the generation of reactive oxygen species (ROS) in and around the tissue.

In step S103 and without being bound by theory, the light source and/or the resulting ROS can modulate cytokine activity. In some embodiments, the cytokine activity includes cytokine expression, for example protein expression of cytokines and/or proteins that regulate cytokine activity (e.g., cytokine receptors). In some embodiments, the cytokine activity includes cytokine signaling, for example, inflammatory signaling. In some embodiments, the laser modulates activity by upregulating or inducing activity. In some embodiments, the laser modulates activity by downregulating or inhibiting activity.

In step S104 and without being bound by theory, the light source and/or the resulting ROS controls tissue remodeling. In some embodiments, the tissue remodeling includes extracellular matrix structural remodeling, cell migration, cell proliferation, cell apoptosis, cell signaling, and the like.

In some embodiments, the laser controls tissue remodeling by locally modifying tissue structure (e.g., protein crosslinking, cell migration, cell-cell interactions including epithelial-stroma interactions, protein-ligand interactions, etc.). As used herein, local and/or locally is assumed to refer to the tissue contacted by the laser and tissue immediately adjacent to that tissue. In some embodiments, local tissue expands to include a margin beyond the tissue directly contacted by the laser, for example about 0.1 μm-10 μm, 10 μm-100 μm, 100 μm-1 mm, 1 mm-10 mm, or 10 mm-100 mm from the tissue directly contacted by the laser.

In some embodiments, the structure of extracellular matrix constituents, e.g., extracellular matrix proteins including collagen and/or collage fibers is remodeled. In some embodiments, the secondary, tertiary or quaternary structure of one or more extracellular matrix constituents is modified and/or remodeled. In some embodiments, the laser controls remodeling by regulating the expression or activity of proteins or enzymes within the extracellular matrix. For example, in some embodiments, the laser regulates the activity of matrix metalloproteinases (MMPs), tissue inhibitors of metalloproteinases (TIMPs), and the like.

In some embodiments, the tissue is tightened. In some embodiments, the tissue is loosened. In some embodiments, the tissue is thickened. In some embodiments, the tissue is thinned. In some embodiments, features in the tissue including ridges, dimples/depressions, wrinkles, laxity, cellulite, and the like, are removed or reduced. In some embodiments, features are generated. In some embodiments, the laser of the disclosed subject matter controls tissue remodeling including controlling cell migration, cell proliferation, cell apoptosis, cell signaling, and the like. In some embodiments, the cells are cells found in a tissue of interest, for example keratocytes, chondrocytes, myocytes, other fibroblasts, epithelial cells, endothelial cells, and the like.

Exemplary Therapies

Embodiments of the disclosed subject matter can be used in various non-ophthalmologic procedures and/or various non-arthroscopic procedures to treat various disorders where decreasing inflammation, scar formation, or cytokine activity is desired (e.g., treatment of disorders of the skin, or other tissues where scar formation or inflammation may have negative cosmetic or functional consequences, including ligaments, tendons, skin, bone, vascular tissue, fascia, cartilage including articular cartilage, or other connective tissue). In some embodiments, the cytokine activity includes chemokine signaling, interleukin signaling, interferon signaling, lymphokine signaling, tumor necrosis factor signaling, and the like. In some embodiments, the interleukin is one or more selected from interleukin 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, and 36. In some embodiments, the interleukin is interleukin 1 and/or interleukin 2. In some embodiments, the interleukin is IL-la. In some embodiments, the interleukin is IL-1β.

The methods and systems can also be used as a prophylactic treatment to prevent unwanted tissue remodeling, e.g., scar formation, scar contracture in implant pockets, and the like. For example, the methods and systems can be used to prevent infiltration of unwanted or excessive fibroblasts that can induce unwanted scars or other unwanted tissue remodeling.

The methods and systems can also be used as a prophylactic treatment to induce wanted tissue remodeling. For example, in some embodiments, the methods and systems are used to control or induce extracellular matrix remodeling.

In some embodiments, the controlled or induced extracellular matrix remodeling controls or directs cell migration and/or proliferation (e.g., promote epithelial cell migration and proliferation, control epithelial-stromal interactions, etc.) and promotes wound healing while reducing unwanted remodeling or scarring (e.g., prevents infiltration of fibroblasts that generate scarring or unwanted tissue composition). This can be especially helpful in the treatment of glaucoma, where remodeling leads to an unwanted closure of the intended filtration path defined by surgery, stent, valve or potentially a laser.

In some embodiments, the methods and systems are used non-prophylactically, that is, the methods and systems are used to treat tissue that has undergone unwanted tissue remodeling, or comprises unwanted tissue structure (e.g., scarring) or composition (e.g., concentration of fibroblasts, epithelial cells, or the like, in a given region). One example could be the remigration of lens epithelial cells under the intraocular lens (secondary cataract formation) after cataract surgery.

The methods and systems can be used to treat various disorders of other collagenous tissues (e.g., skin, tendons, ligaments, neural tissue, vascular tissue, cartilage, fascia, muscle, bone, and the like) including scarring.

Exemplary Irradiation Parameters

As described in International Publication No. WO 2017/070637 and U.S. Patent Application Publication Nos. 2018/0193188 and 2018/0221201, tissue treatment can be achieved without the need for exogenous photosensitizers such as riboflavin by ionizing water within ophthalmologic (e.g., corneal) or other tissue to generate reactive oxygen species. Treatment can be achieved over a broad range of wavelengths including those that are not absorbed by amino acids within collagen strands. For example, the laser wavelength can be in the range from about 250 nm to about 1600 nm, e.g., 780 nm, 1030 nm, 1040 nm, and the like. In some embodiments, the laser wavelength is between laser wavelength can be in the range from about 250 nm to about 1600 nm, but excluding wavelengths between 260-290 nm, 520-580 nm, 780-870 nm, and 1040-1160 nm.

By controlling pulse energy to be below the optical breakdown threshold of water or collagen (about 1.0×10¹³ W cm⁻²), the effects disclosed herein can be modified without modifying the refractive index of the collagen.

Ionization can be created within tissue using a laser emission that is absorbed by the tissue. For example, the laser emission can be based on ultrashort laser pulses. As used herein, the phrase “ultrashort laser pulses” includes emissions in the femtosecond, picosecond, and nanosecond ranges. Nonlinear absorption of laser emissions can occur, in part, due to the highly compressed nature of the light pulses, allowing treatments of the interior of a biological dielectric, such as collagenous tissues including corneal tissue and cartilage, without affecting the surface layer. The dielectric can be transparent for the wavelengths applied (e.g., infrared).

The ultrashort laser pulse can induce low-density plasma that ionizes water molecules within the tissue, while still operating below the energy level required for optical breakdown. Optical breakdown is the effect of an ultrafast laser focused in the interior of collagen-rich tissue, where photoionization triggers non-linear absorption. Continued supply of incoming photons leads to the buildup of free electrons, further leading to avalanche ionization, which enhances the growth of free electron density resulting in formation of plasma. As contrasted from the low-density plasma, high-density, opaque plasma strongly absorbs laser energy through free carrier absorption. The high-density plasma expands rapidly, creating a shock-wave that propagates into surrounding material, creating optical breakdown.

The effects disclosed herein can be safely induced when the laser is operated below optical breakdown level in the so-called “low-density plasma” regime. For example, the laser emission, as defined by its wavelength, temporal pulse width, and pulse energy, as well as the numerical aperture of the scanning objective and the scanning speed should be high enough to induce ionization of water molecules in the collagen rich tissue, but below optical breakdown level. Further, such ionization can be induced in the cornea without reducing the transparency of the cornea or other tissue.

Without being bound by theory, the ionization can cause the formation of reactive oxygen products, such as singlet oxygen, OH⁻, and H₂O₂. Singlet oxygen generated by the ionization can inactivate collagenase and have a germicidal effect, increasing the utility of these methods for clinical applications. In embodiments, deuterium oxide can be introduced onto or into the tissue to prolong half-life of the produced singlet oxygen, thereby increasing efficiency.

In certain aspects, the disclosed subject matter provides methods of inducing such ionization. The methods can be used in the treatment of various ectatic diseases or during refractive surgery. The methods can include modulating cytokine activity. In some embodiments, modulating cytokine activity directs cellular behavior, for example migration, proliferation, apoptosis, signaling, and the like, and drives tissue remodeling.

Exemplary System

In some embodiments, the treatment system includes an objective. The objective can be high-magnification lens (e.g., 40×).

The objective can be a scanning objective with a large numerical aperture. The large numerical aperture (NA) allows the objective to focus diffuse light to a small area. A laser supplies the light (e.g., laser light) to the objective. In one embodiment, the NA is 0.4. In another embodiment, the numerical aperture is 0.6, with a long working distance. In another embodiment, the numerical aperture is between 0.05 and 1.4. However, the NA could be varied together with the pulse energy to achieve similar effect in a different control volume. Without being bound by theory, Applicant believes that NAs can be varied between about 0.05 and about 1.4, and paired with an appropriate pulse energy in order to generate low-density plasma without inducing or causing optical breakdown. In one embodiment, NAs between about 0.4 and about 0.95 would be capable of creating low-density plasma without causing optical breakdown.

In an embodiment, one or more optical filters can be interspersed between the laser and the objective.

The laser can be a femtosecond laser that outputs laser light. In some embodiments, the laser light has a single frequency, multiple frequencies, or a spectral bandwidth. Embodiments can use any wavelength including multiple or continuous spectra covering a wide range of wavelengths. In embodiments, radiation at frequencies that may harm tissue or reduce the locality of the generation of reactive species are minimized or eliminated. Radiation that may be directly absorbed by the collagen can be minimized or eliminated. In an embodiment, the frequency or frequencies of the laser are outside of the ultraviolet range. In embodiments, the frequency or frequencies of the laser are in the infra-red frequency band. The laser receives control input from controls, which can be implemented on a stand-alone processing device or as embedded circuitry of the system.

Generation of such short pulses can be achieved with the technique of passive mode locking. The laser can be any suitable laser type, including bulk lasers, fiber lasers, dye lasers, semiconductor lasers, and other types of lasers. In an embodiment, the laser operates in the infrared frequency range. In other embodiments, the lasers may cover a wide range of spectra domain. In embodiments, the disclosed subject matter can be implemented as an add-on system to a femtosecond laser system, such as used in certain Lasik systems.

In particular embodiments, the laser can be a Nd:Glass femtosecond laser. In embodiments, the laser wavelength can be in the range from about 250 nm to about 1600 nm. In embodiments, the femtosecond laser can have a temporal pulse width of from about 20 fs to about 26 ps. In embodiments, the pulse energy is from about 0.1 nJ to 100 nJ, 0.1 nJ to about 50 μJ, 0.1 nJ to about 10 μJ, from about 0.5 nJ to 50 nJ, or from about 1 nJ to 10 nJ.

In embodiments, the femtosecond laser can be a SPIRIT® femtosecond laser in combination with a SPIRIT-NOPA® amplifier (Spectra-Physics, Santa Clara, Calif.).

In some embodiments, the objective focuses incoming laser light into a focused beam that irradiates a target. In some embodiments, the target is corneal tissue. In some embodiments, the target is other collagenous tissue. The objective may have a large numerical aperture.

In some embodiments, the system includes an imaging system that includes controls, which can communicate with controls of the treatment system. The imaging system can implement en face imaging, tomography, topographic imaging, and the like. These sub-systems or a combination thereof can be used to guide the treatment and allows better treatment options and guidance.

The imaging system can include a light source and an imaging device, such as a camera. The light source projects light to mirror and a device, such as a mask, to produce an illumination pattern. The illumination pattern guides the system to produce the desired change in the treated tissue. The desired change may include changes in local cytokine activity.

In embodiments, multiple beams can be provided by splitting a laser beam into multiple spots, for example, by use of a phase plate or other means to spatially separate the focus. For example, a laser head can include multiple scanning objectives bundled together. A high energy laser beam (e.g., having a pulse energy of greater than about 10 μJ) can be split using a beam splitter to send individual laser beams to each scanning objective. Therefore, the number of passes required to fully treat the cornea can be reduced by providing multiple laser beams simultaneously. In embodiments, an entire corneal layer could be treated simultaneously, e.g., by bundling many scanning objectives to the laser head such that only one pass is required.

Generation of ROS

In some embodiments, the modulation of cytokine activity and/or prevention or decrease of inflammation, scar formation, or cytokine activity is achieved with alternative approaches for generating ROS. In some embodiments, ROS is generated by low density plasma in aqueous solutions, as described herein. In other embodiments, ROS is generated by other physical means of ionizing a tissue. For example, in some embodiments, ROS is generated using one or more of UV light, X-ray, other suitable electromagnetic wave, plasma gun, nanosecond laser, or other means as understood by one skilled in the art. In some embodiments, ROS is generated by chemical means. For example, in some embodiments, ROS is generated using one or more of hydrogen peroxide, ascorbic acid, saponins, xanthine oxidase/hypoxanthine, tert-butyl hydroperoxide, sodium hypochlorite, and the like, as understood by one skilled in the art.

Implementation in Computer-Readable Media and/or Hardware

The methods described herein can be readily implemented, in whole or in part, in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### WORKING EXAMPLES

### Working Example 1—Treatment of Ex Vivo Porcine Eyes

A total of 60 fresh pig eyes were used for the study. Fifteen of these eyes underwent corneal flattening, and the treated eyes were paired with 10 control eyes. Thirteen eyes underwent laser irradiation to induce post-treatment steepening; these eyes were also paired with 10 control eyes. The remaining 12 eyes were used for a separate control study, to evaluate the effects of the experimental setup.

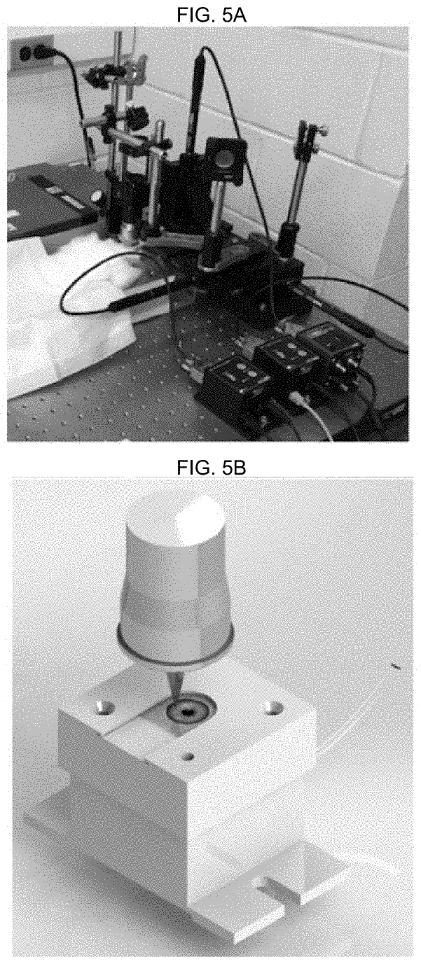

Excess tissue was removed from the isolated eyes, which were then rinsed with Dulbecco's phosphate-buffered saline (DPBS, 1×, Sigma-Aldrich) and brought to room temperature in a damp chamber. The eyes were examined and any defective samples were discarded. The epithelial layer of the retained specimens was removed by gentle scraping with a 10 mm scalpel blade and the eye globes were mounted on a custom-built eye holder (FIG. 5A).

The epithelial layer was removed to ensure consistency between the specimens, as most had experienced superficial damage during handling at the abattoir. Intraocular pressure was maintained (˜16 12 mm Hg) by connecting an intravenous (IV) system filled with 0.9% sodium chloride solution (Hospira Inc, Lake Forest, Ill.) to the eyeball via a 22 G needle (BD, Franklin Lakes, N.J.). A customized digital pressure gauge with an OMEGA™ PX154 low-differential pressure transmitter was used to adjust the pressure. Immediately before treatment, the corneal surface was covered with a microscope coverslip (#1 Microscope Cover Glasses, VWR International, PA) to ensure homogeneous volumetric irradiation of the cornea.

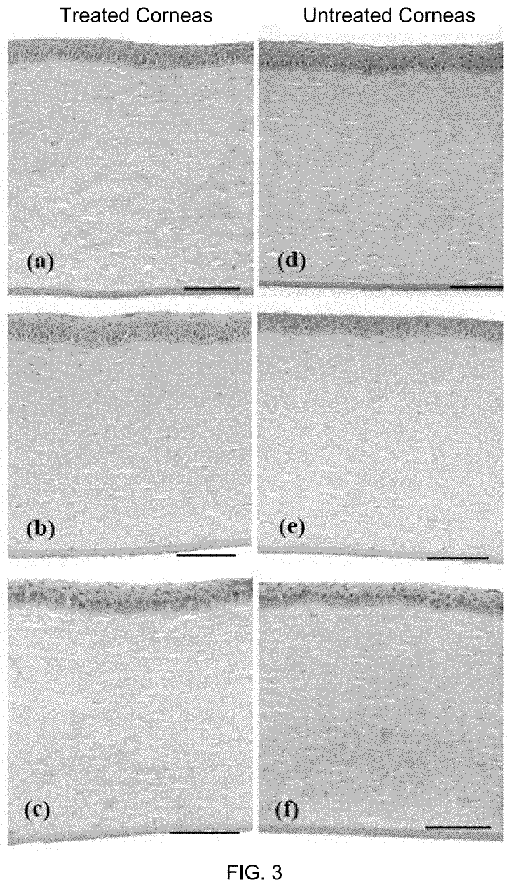

An Nd:Glass femtosecond oscillator (High Q Laser, Austria) was used to generate laser pulses with a temporal pulse width of 99 fs and a repetition rate of 52.06 MHz at a wavelength of 1059.2 nm. A ZEISS® PLAN-NEOFLUAR® 40× objective lens with a numerical aperture (NA) of 0.6 was used to focus the beam, and the mean power of the laser system after the objective lens was 60 mW. The samples were mounted on a three-axis motorized PT1 translational stage powered by Z825B motorized actuators (Thorlabs, Newton, N.J.). The treatment consisted of laser pulses applied by moving the stage in an x-y plane, such that the laser path followed a zigzag trajectory at a feed rate of 2.2 mm/s, resulting in the treatment of a planar area at a specific depth. The objective focused the laser on a spot of about 2 μm in diameter. There was, therefore, a spot overlap of about 90%. The treatment was repeated at different depths, resulting in ‘treatment layers’. Multiple treatment layers were created parallel to the surface, with a distance of 50 μm between two consecutive planes, giving an effective depth of treatment of about 200 μm. The laser beam was focused on the anterior cornea, from the corneal surface to a depth of up to 200 μm. The experimental set-up is shown in FIGS. 5A and 5B, and schematic diagrams of the treatment paths are shown in FIG. 3, Panels A, B, and C.

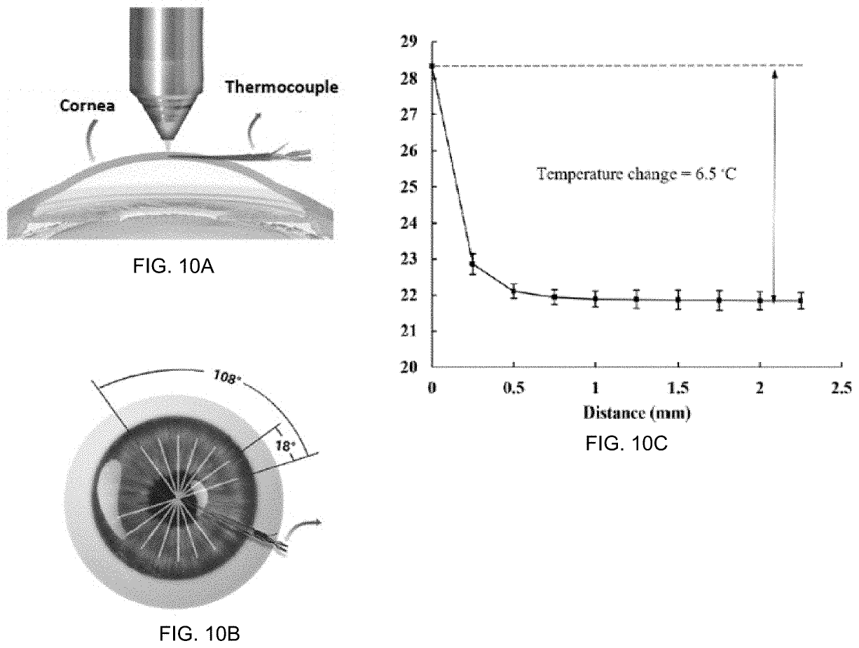

For confirmation that the induced changes were photochemical in nature, with no influence of the thermal denaturation of collagen fibrils, Applicant measured the laser-induced changes in corneal temperature. The relative change in temperature at the focal volume and in its immediate vicinity was less than 7° C. The heating induced by the treatment was, therefore, well below the threshold for the thermal denaturation of collagen. (FIG. 10).

Specifically, the needle-like tip of a customized thermocouple (Omega Single Strand, Insulated Thermocouple Wire with a 0.07 mm diameter, temperature measurement range 0-100° C., Stamford, Conn.) is inserted into the middle of the cornea, parallel to the surface. The focal point is carefully aligned with the tip of the thermocouple and temperature distribution is measured as the focal volume is moved laterally away from the tip of the thermocouple. Each consecutive temperature measurement is made along a testing line 18° away from the previous measurement, in the same plane.

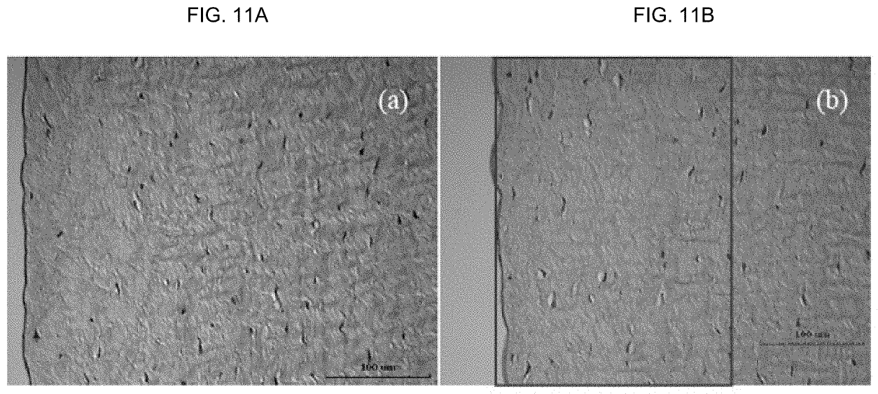

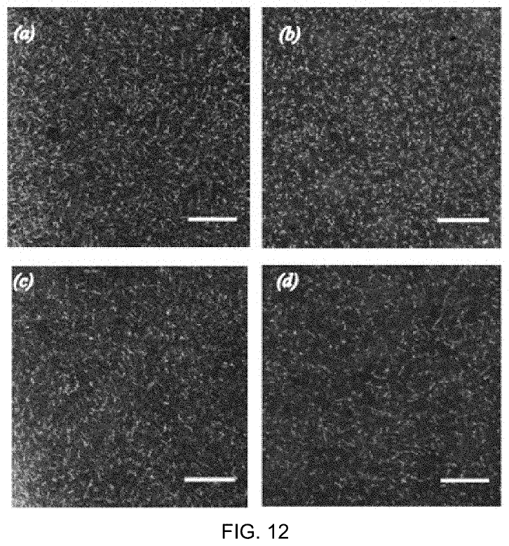

Furthermore, Nomarski interference contrast characterization is performed on corneas, 24 hours after treatment. A Nomarski interference contrast (refractive index sensitivity of about 0.08) prism is used to enhance the contrast between regions of the cornea with potentially different refractive indices. Before the examination, corneas are fixed by incubation in 10% formalin overnight, followed by desorption in 70% alcohol for 24 hours. Light microscopy with a microscope equipped with Nomarski interference contrast optics revealed no difference in refractive index between the treated and untreated parts of the cornea, consistent with an absence of corneal hazing (FIGS. 11A-11B).

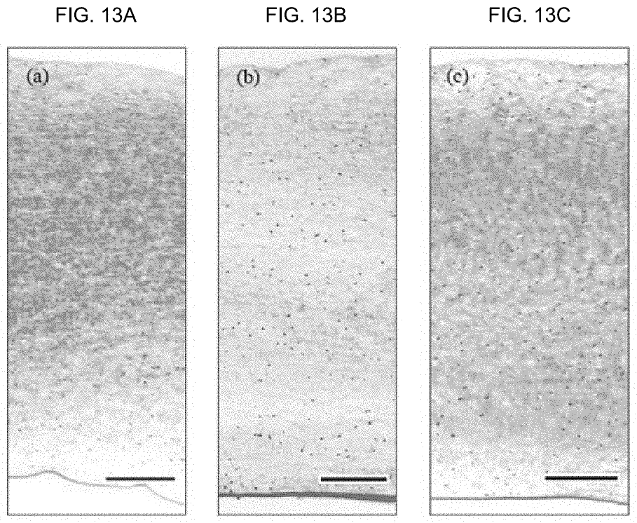

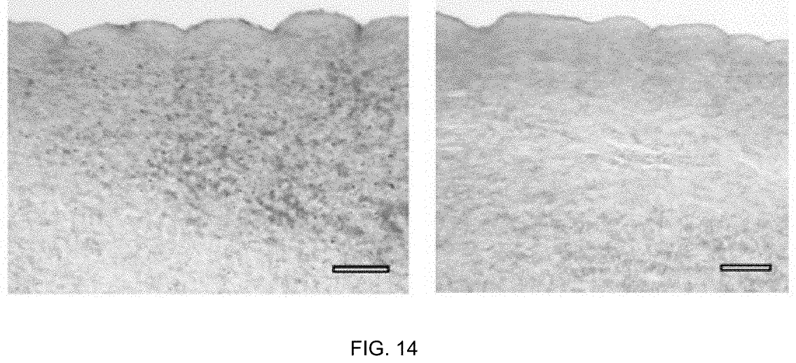

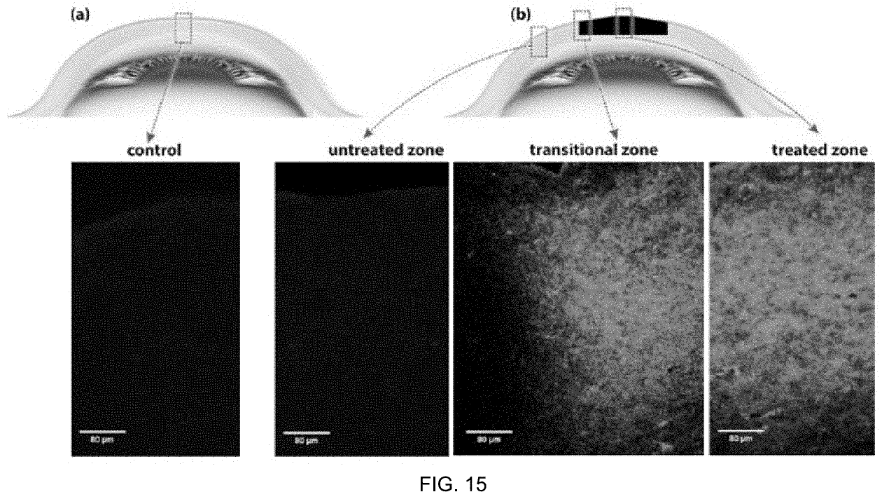

Standard histological examinations of the pig corneas were also performed. No laser-induced damage was observed on hematoxylin-eosin (H&E)-stained histological sections of corneas (FIGS. 13A-13C). Treatment was found to delay the injury-induced apoptosis of stromal keratocytes (FIG. 14).

### Working Example 2—Spatially Resolved Alterations of Rabbit Eyes

Rabbit models were used to assess the stability of the changes in corneas subjected to laser treatment and the safety of the procedure. A protocol almost identical to that used on pig eyes is applied to rabbit eyes in vivo, with a view to assessing changes in effective refractivepower (ERP), 24 hours, seven days, and then weekly up to three months after treatment. Three groups of animals were assessed. The animals in the first group (n=3) were euthanized and their eyes were removed 48 hours after laser treatment, to investigate the acute effects of laser irradiation. The animals in the second group (n=3) were euthanized after one week, to allow the eyes to undergo at least partial healing if the laser had damaged the tissue. In this group, the refractive power of the eye was also determined 48 hours after treatment. The last group of animals (n=6) was monitored to investigate the long-term stability of the induced changes in refractive power. Half of the animals in the last group were euthanized after three months to assess whether there were any treatment-induced damage or other morphological changes. The animals were euthanized 48 hours (group 1), seven days (group 2), and three months (half of the animals in group 3) after treatment, by the intravenous injection of pentobarbital (100 mg/kg) into the marginal ear vein. These changes remained stable for three months after treatment, with a relative change in EPR of about 1.94 diopters for treated eyes.

Ex Vivo Measurements

Ex vivo corneal topography characterization was performed using an EYESYS VISION™ clinical eye topography characterization instrument (EYESYS® System 2000, EyeSys Vision Inc, TX) with version 1.50 software to capture corneal topography and calculate effective refractive power immediately before treatment, and after laser irradiation. Measurements were made at regular intervals over a 24-hour period after treatment of the eyes. During this characterization, the corneal surface was moistened with SYSTANE ULTRA® Lubricant Eye Drops (Novartis). Following the application of an eye drop, the eye holder was moved gently in a circular motion, to distribute the lubricant evenly over the ocular surface and allow any excess lubricant to slide off the eye.

The structure of hematoxylin-eosin (H&E)-stained histological sections of corneas obtained 48 hours, one week and three months after treatment was similar to that of the control samples (FIG. 3). The laser treatment therefore caused no damage to the epithelial, stromal, and endothelial layers. Specifically, no wound or wound healing response resembling that observed after refractive surgery was detected, and no collagen disorganization, epithelial cell and stromal edema, intrastromal vacuole formation or endothelial cell detachment was observed; all these features are associated with thermal damage to stromal tissue, and therefore it has been determined that no such damage occurred.

Second-Harmonic-Generation Microscopy

Corneas were harvested immediately after laser irradiation of the eyes. They were fixed by incubation overnight in 4% paraformaldehyde at 5 C, and then 2 mm² blocks were dissected from the central region, washed in PBS, mounted in 50% glycerol in PBS on microscope coverslips and imaged. The second harmonic signal was generated with a laser (CHAMELEON VISION® II, Coherent, Santa Clara, Calif.) tuned to 850 nm, on an A1RMP laser scanning system mounted on an ECLIPSE® TiE microscope stand (Nikon Instruments, Melville, N.Y.) equipped with a 25×/1.1 NA ApoLWD water-immersion objective. The back-scatter configuration was used to acquire the SHG signal, with the non-descanned detector (Nikon, Japan) and a 400-450 nm bandpass filter. The images were preprocessed with the spatial frequency filter. Set-ups with 100 and 20 pixels were used to analyze large and small features, respectively. After initial processing, the images were converted to binary signals, and plotted. The optical density of the black and white areas on the binary images was assessed by determining the number of peaks crossing the median cut-off intensity. The crossing densities measured at the vertical midline are considered to represent the complexity (irregularity) of collagen structure patterns: the higher the crossing density, the more complex the collagen bundle pattern. SHG imaging and analysis were performed on the central regions of anterior corneas. Groups of laser-treated and control samples were prepared for SHG imaging, with each group containing four corneas.

Differential Scanning Calorimetry (DSC)

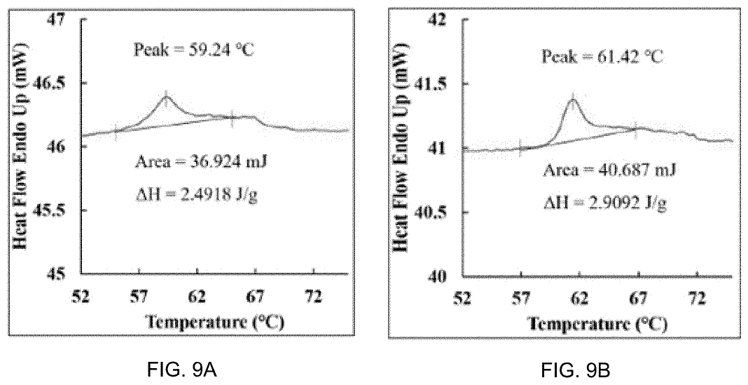

Corneal punches of 5 mm in diameter were extracted from laser treated and untreated corneal samples, sealed in plastic film to stop the tissues from drying out and stored frozen at −20 C. The punch samples were loaded into the DSC autosampler (PERKIN-ELMER DSC 6000 autosampler) for the measurement of denaturation temperature. Samples were heated to temperatures of 40 C to 70 C, at a scanning speed of 18 C/min. Denaturation curves representing differential heat flow over time were generated (FIGS. 9A and 9B) and analyzed with PYRIS™ software (version 11.0).

Two Photon Fluorescence (TPF) Microscopy

Isolated untreated control and laser treated corneal samples were cut into 2 mm² blocks by a customized slicer and mounted in a 3 mm Petri dish. TPF was conducted by a two-photon microscope (Bruker) with MAI TAI™ DEEPSEE™ Ti:Sapphire laser (Spectra Physics) as the excitation source. A 40×/0.8 NA water immersion objective (Olympus) was applied to collect the fluorescence signal. The signal was registered with two different photomultiplier tubes, one in the red (580-620 nm) and one in the green (480-570 nm) wavelength regime. Excitation wavelengths used were 826 nm to excite collagen matrix.

In Vivo Measurements

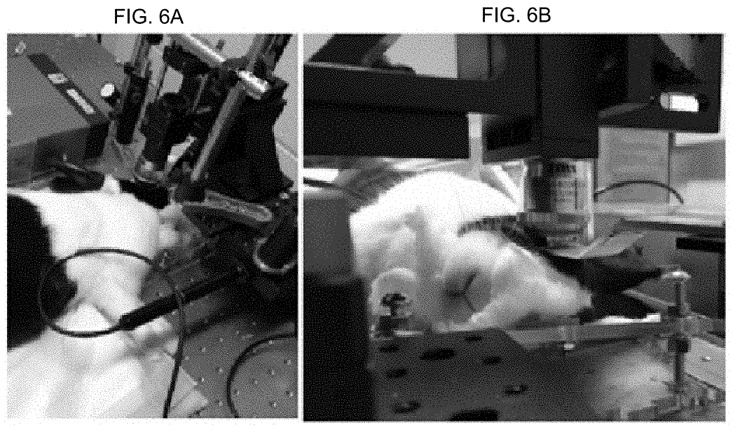

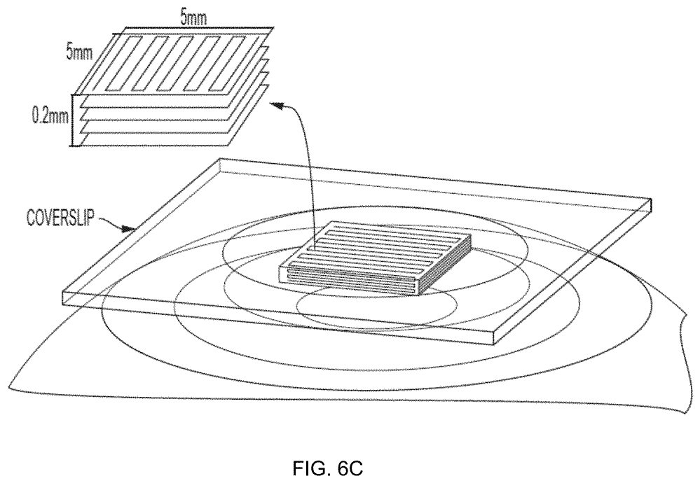

In vivo measurements were obtained in anesthetized rabbits using a custom-built, heavily padded holder for immobilization (FIGS. 6A and 6B). The eye facing upwards was treated, and the eye facing downwards was used as the untreated control. The treated eye was gently pressed with a cover slip to ensure the homogeneous volumetric application of laser pulses. The laser treatment protocol was based on the procedure developed on porcine eyes ex vivo, as described above. However, the laser beam (Nd:Glass ultrafast laser, Hi-Q Laser, Austria) focused, via an objective with a high numerical aperture (Zeiss, PLAN-NEOFLUAR® 40×/0.6 NA), on the desired volumetric zone of the cornea was delivered to the rabbit eye by mounting the objective on a custom-built three-axis motion system with three translational stages (PT1, Thorlabs, Newton, N.J.) coupled to motorized actuators (Z825B, Thorlabs, Newton, N.J.). A number of optical components, including mirrors and lenses, were mounted on the motion system, to steer the laser beam into the back aperture of the objective. As in the ex vivo study, laser pulses were rasterized by moving the objective in an x-y plane such that the laser beam followed a zigzag trajectory, resulting in the treatment of a circular planar area (5 mm diameter) at a specific depth (FIG. 6C). Again, as in the ex vivo study, the treatment was repeated at different depths, to generate ‘treatment layers’. Five treatment layers parallel to the surface were created, with a distance of 50 μm between consecutive layers.

Confocal Laser Scanning Microscopy (CLSM)

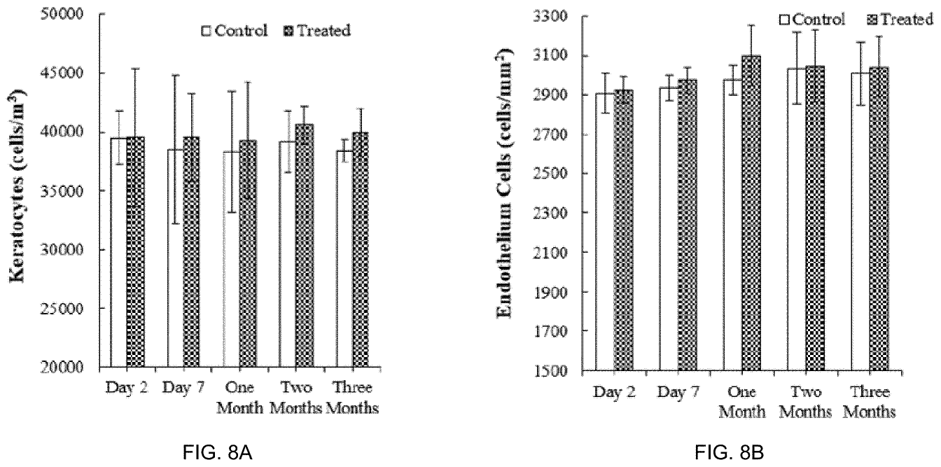

Referring now to FIG. 11A-11E, CLSM was employed for cellular evaluations of corneal tissues. CLSM imaging was performed with the HRT3-RCM laser scanning system (670 nm laser beam, Heidelberg Engineering) equipped with a 63×/0.95 NA water immersion objective (Zeiss). Imaging was performed immediately after the rabbits were euthanized for group 1 (48 hours after treatment) and group 2 (7 days after treatment), and in vivo for group 3 (one, two, and three months after treatment). A disposable sterile plastic cap was placed on the objective to maintain the distance between the corneal surface and the objective. The animals were placed in a custom-built holder during the process, with the eyelids of the imaged eye gently pulled open by hand. GENTEAL™ water-based gel was applied as a coupling medium. The entire corneal volume was scanned and recorded, with optical sections through the epithelium, stroma, and endothelium. Rabbit corneal keratocyte and endothelial cell densities are shown in FIGS. 8A and 8B.

The distance between two consecutive image planes was 2 μm for the epithelium and the stromal keratocyte network. The monolayer of endothelial cells was imaged separately. A comparison of CLSM images for intact rabbit eyes and laser-treated rabbit eyes revealed no significant difference in cellular structure. Laser irradiation did not, therefore, damage cellular components. The CLSM images of the endothelium demonstrate that cell shape and density were similar for treated eyes and their paired controls.

### Working Example 3—Spatially Resolved Alterations of Cultured Pig Corneas

Pig corneas were cultured after treatment, to assess the effects of femtosecond oscillator irradiation in order to determine whether any degradation occurred in the crosslinked layers of the stromal matrix and whether there were any adverse effects on cellular components. Cultured corneas were evaluated on days 1 and 7.

Methods

Freshly harvested pig eyes were treated with the femtosecond oscillator, according to the procedure outlined in the methods section. A total of 40 eyes were evaluated: 20 of these eyes were treated, and the other 20 were paired controls. After laser treatment, the corneal topography of the eyes was monitored for 24 hours under controlled conditions. After the topography observation period, eyes were removed from the custom-built eye holders, rinsed three times with 20 ml of sterile phosphate-buffered saline (PBS) per rinse, immersed in 20 ml of 3% polyvinylpyrrolidone-iodine (PVP-I) solution for about 1 minute and rinsed again three times with sterile PBS. After the last rinse, corneas were dissected from the eye together with about 1 mm (thickness) of the scleral rim. Half of each sample was immediately examined (24 hour time point), and the other half was placed in a sterile culture vessel (Fisher Scientific, CAT #08722E). The vessel was filled with 8 ml of a customized incubation medium consisting of low-glucose Dulbecco's Modified Eagle Medium (Thermo Fisher) supplemented with 8% fetal bovine serum and the appropriate antibody. Corneas were cultured at 37° C. in a tissue culture incubator (Thermo Scientific Series 8000 DH, Waltham, Mass.). Cell viability (LIVE/DEAD™ Assay Kit, Invitrogen) in the corneal stroma was assessed for both the 24-hour and one week after treatment time points. Cylinders having a 5 mm-diameter were punched out from the central part of with the cornea for confocal microscopy assessment (Olympus Fluoview FV1000, Waltham, Mass.). The images were taken from the anterior side of the corneal samples, in the middle part of the sample thickness. In addition to cell viability tests, standard histological characterization was performed on corneas 24 hour and one week after treatment. Corneas were fixed by incubation with 10% formalin overnight, desorbed by incubation with 70% alcohol for 24 hours and were embedded in paraffin wax, and cut into 5 μm sections, which were stained with hematoxylin and eosin (H&E).

Results and Discussion

Confocal images of samples stained with LIVE/DEAD™ assay kit provided no evidence of a loss of viability 24 hours after treatment (FIG. 12, Panels (a) and (b)). Similarly, no loss of viability relative to the untreated control was observed for the specimens cultured for one week after treatment FIGS. 13A-13C. These results demonstrate that the laser treatment does not affect cell viability for at least week, as no significant post-treatment keratocyte depopulation was observed in the corneal stroma of treated samples.

However, qualitative observations of the control and post-treatment confocal micrographs (FIG. 12) revealed that cell density was higher in treated than in control specimens. This observation was consistent with the findings for H&E-stained slides of tissue (FIGS. 13C and 14). H&E-stained corneal sections of the femtosecond laser-treated eyes displayed no significant change in stromal structure and endothelium integrity relative to the untreated control (FIGS. 13A-13C), but the treated anterior segments of the corneas remain populated with keratocytes 24 hours after treatment, and after culture in tissue culture medium for 7 days at 37° C. (FIG. 14). After one week of culture, keratocytes were still present, throughout the entire cross-section, in the treated samples (FIG. 13C), whereas they were present only in the posterior regions of cultured control corneas (FIGS. 13A and 14).

This finding was unexpected, as debriding of the corneal epithelium results in apoptosis-driven keratocyte depopulation. In this study, the corneal epithelium was removed because most samples suffered superficial damage due to handling at the abattoir. It was therefore expected to find an absence of keratocytes in the anterior stroma. Previous reports suggested that epithelial-stromal apoptosis serves as an antiviral response mechanism, limiting the proliferation of pathogens, such as herpes simplex virus, from the injured corneal epithelium to the stroma. A similar disappearance of keratocytes from the anterior stroma has been observed after photorefractive keratectomy (PRK), in which scraping of the epithelium is followed by excimer laser-assisted photoablation of the corneal stroma to correct refractive errors. It has been suggested that damaged epithelial cells release interleukin-1 (IL-1) into the corneal stoma, regulating keratocyte apoptosis. In normal homeostasis, IL-1 maintains tissue organization through apoptotic and, possibly, negative chemotactic effects on adjacent keratocytes. However, its effect on individual keratocytes depends on its local concentration and, if it remains below lethal concentrations, the keratocyte will respond by negative chemotaxis rather than apoptosis.

In the experiments presented, the epithelium was scraped off the pig eyes before laser treatment, which should have triggered the release of IL-1 into the corneal stroma. However, no major keratocyte apoptosis was observed in the anterior stroma. Without being bound to any particular theory, results suggest either propagation of the IL-1 signal was retarded by the treatment or that cell-cell interactions were altered.

Histological examination of the treated cornea showed that the proposed laser treatment had no adverse effects on the stromal tissue. No collagen disorganization, stromal edema, intrastromal vacuole formation or endothelium detachment was observed. Furthermore, the treatment appeared to retard the injury-induced apoptosis of stromal keratocytes. Corneal fibroblasts in keratoconic eyes have four times as many IL-1 receptors than those of normal eyes. In normal homeostasis, IL-1 balances keratocyte proliferation and apoptosis.

### Working Example 4—Ex Vivo Experiments on Rabbit Eyes

The cornea is comprised of three major cell-containing layers: an external layer of epithelium, an extracellular matrix—rich stroma, and an endothelial monolayer. The stroma is populated with keratocytes—the multifunctional cells responsible for proper maintenance of the corneal collagenous structure and other corneal functions

Wounding of corneal epithelium initiates the rapid loss of the stromal keratocytes. The response is suggestive of epithelial-stromal interactions and attributes a great practical significance, as it plays a central role in precise regulation of the functions conducting wound healing.

The injury triggers upregulated secretion of IL-1 (a, (3) by corneal epithelium, which in their own term induce apoptosis of stromal keratocytes located near the wound. The keratocytes surrounding the apoptotic area differentiate to fibroblasts, and along with inflammatory cells migrate into the wound, secreting proteases, chemokines and growth factors. The transforming growth factor β1 (TGFβ1) stimulates fibroblasts differentiation into myofibroblasts producing stromal extracellular matrix (ECM) components. Excessive collagen deposition can lead to the loss of structural integrity of the ECM resulting in corneal haze, scaring and after refractive surgery regressions.

The conditions associated with an injury and causing a failure of tissue remodeling have been observed in numerous other tissues, including the lens, liver, kidney, and lung. Healing of corneal wound is unique. For most other tissues, the process culminates in vascularization and scar formation—the end points that must be minimized during the cornea healing in order to prevent serious visual consequences.

Development of new technologies for controlled, non-invasive modulation of epithelial-stroma interactions could greatly contribute to novel clinical applications targeting the post trauma and post refractive surgery complications.

Methods

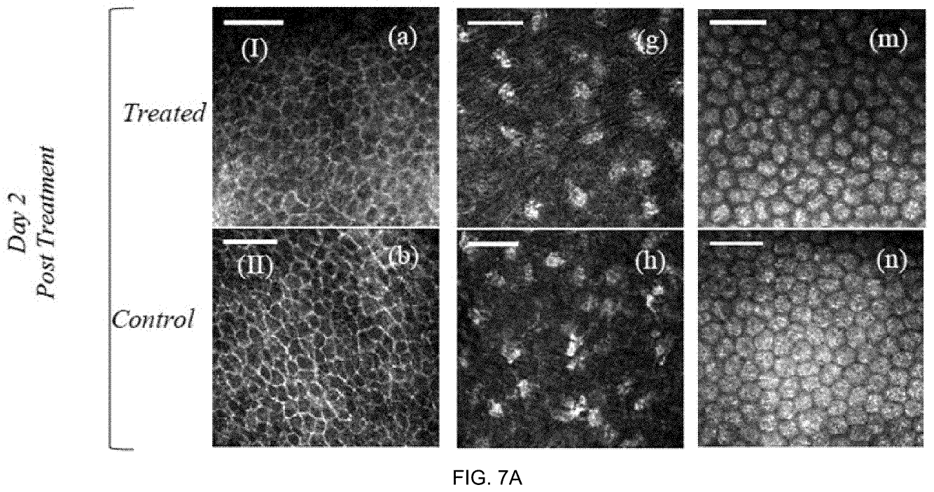

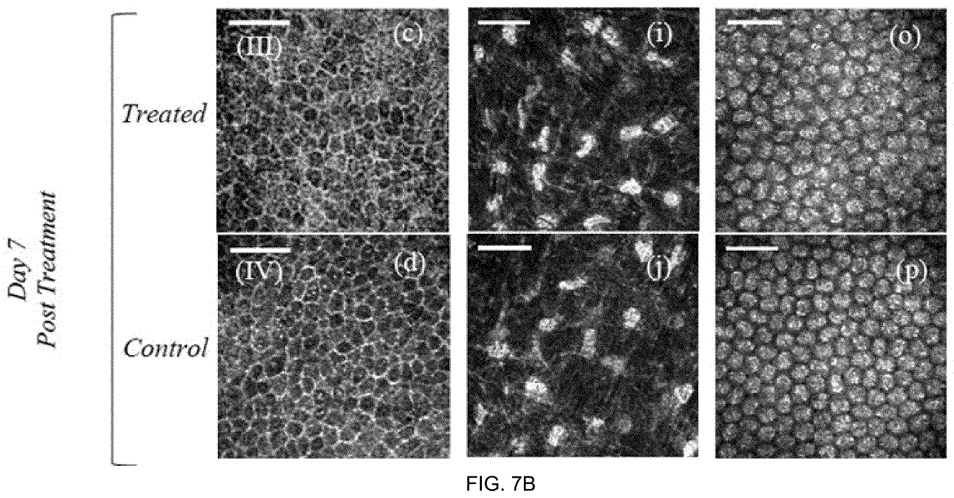

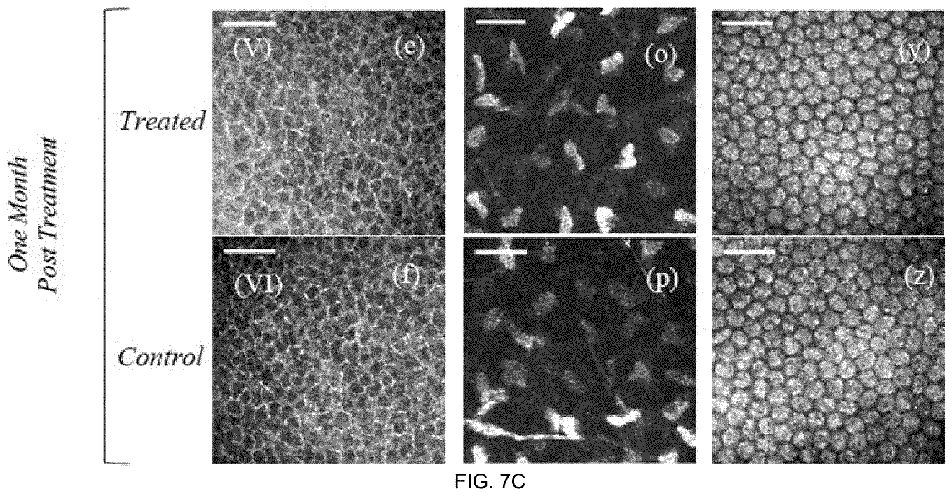

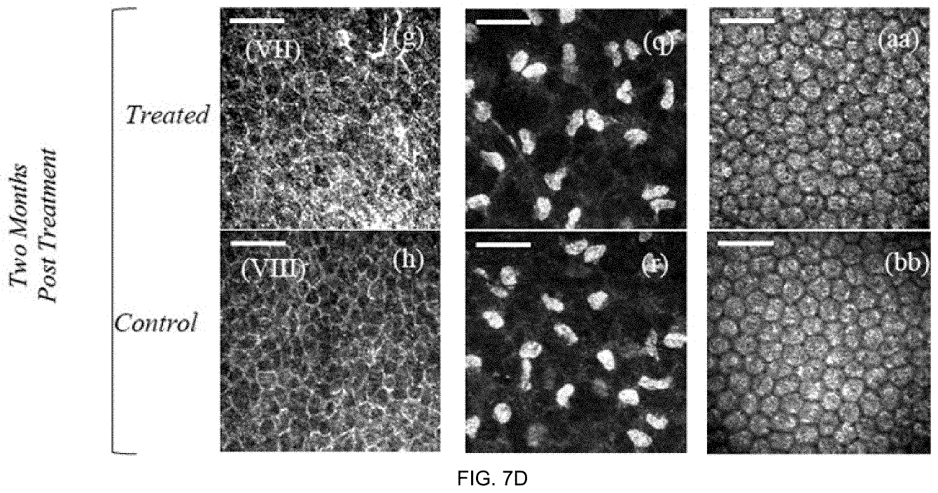

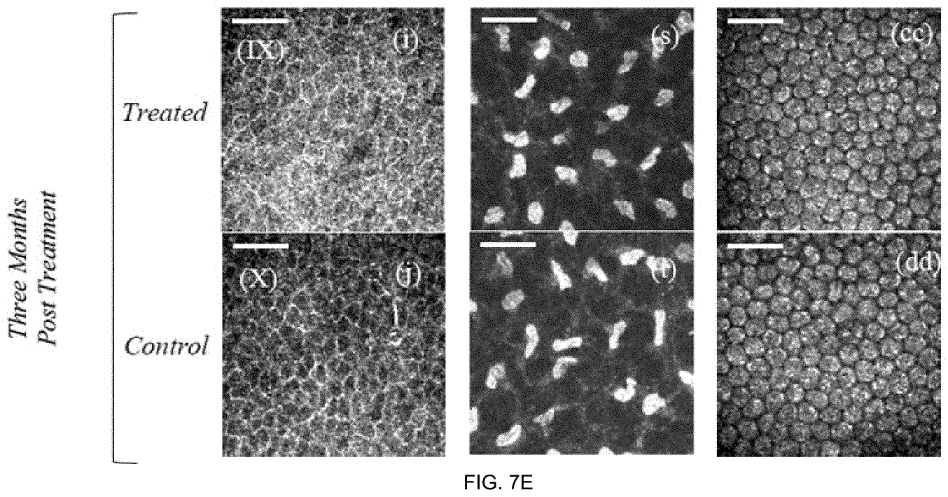

Referring now to FIG. 7A-7E, CLSM was employed for cellular evaluations of corneal tissues. CLSM imaging was performed with the HRT3-RCM laser scanning system (670 nm laser beam, Heidelberg Engineering) equipped with a 63×/0.95 NA water immersion objective (Zeiss).

Results

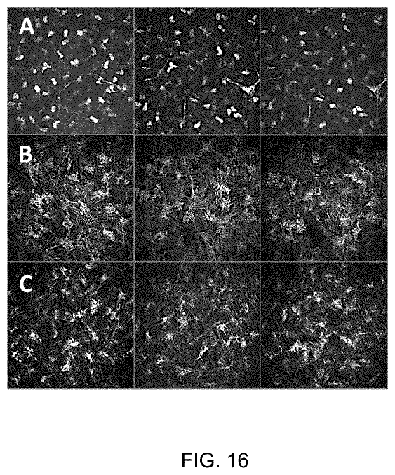

Laser Scanning Confocal Microscopy (LSCM) of Stromal Keratocytes LSCM images of the processed eyes collected at the 24-hour time point, shown in FIG. 16, reveal some noticeable changes in keratocytes networks of the corneas undergoing epithelium debriding for both the laser-treated group (Row C) and untreated group (Row B) compared to the control (Row A).

Quantitative Analysis of the LSCM Images of Stromal Keratocytes

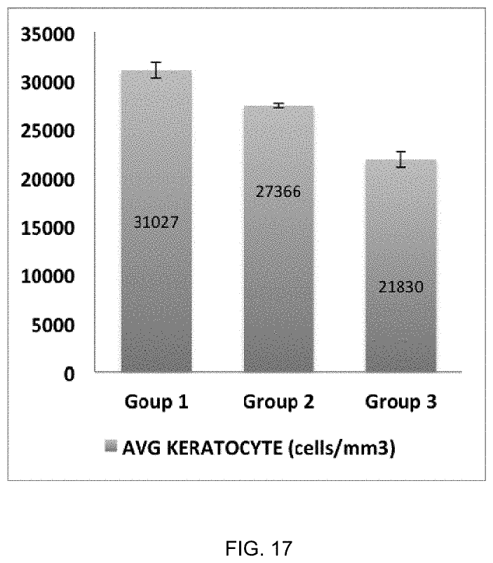

The keratocyte density was averaged across all corneas in each group. Results were graphed and are shown in FIG. 17. At the 24-hour time point, the keratocyte density in the epithelium-debrided and laser-treated corneas (Group 2) is about 12% lower than in the intact control corneas (Group 1). The keratocyte depopulation of the epithelium-debrided corneas that were not laser treated (Group 3) can reach over 30% respectively.

LSCM of Epithelial Monolayers

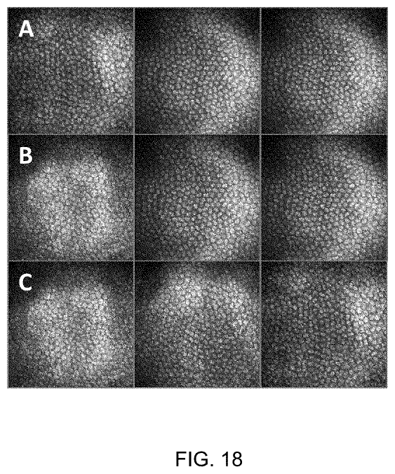

LSCM images of the process eyes collected at the 24-hour time point, shown in FIG. 18, reveal no visible changes in the integrity of the corneal endothelium caused by the epithelium debriding and subsequent laser treatment, under the conditions of the current experiment.

Quantitative Analysis of the LSCM Images of Corneal Epithelium

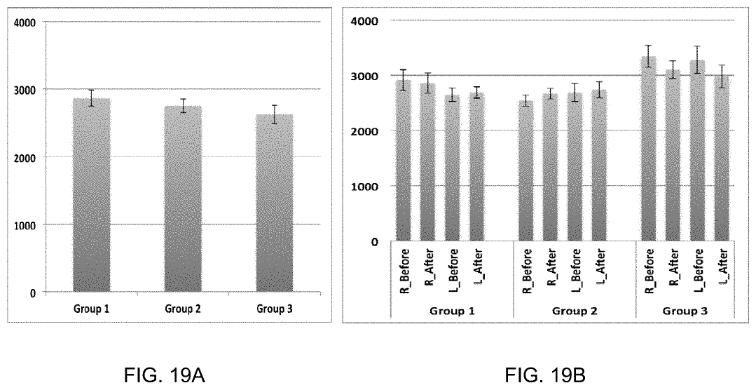

The endothelial cell count (cells/mm²) was averaged and graphed in FIGS. 19A and 19B. Quantitative analysis of the LSCM images of the endothelial monolayers of all tested eye groups shows some decreasing in the average cell number in Group 3 relative to Group 1 and Group 2, shown in FIG. 19A. However, this change in Group 3 relative to Groups 1 and 2 is not statistically significant. FIG. 19B depicts the raw data.

In Vivo Experiment on Dutch Belted Rabbits

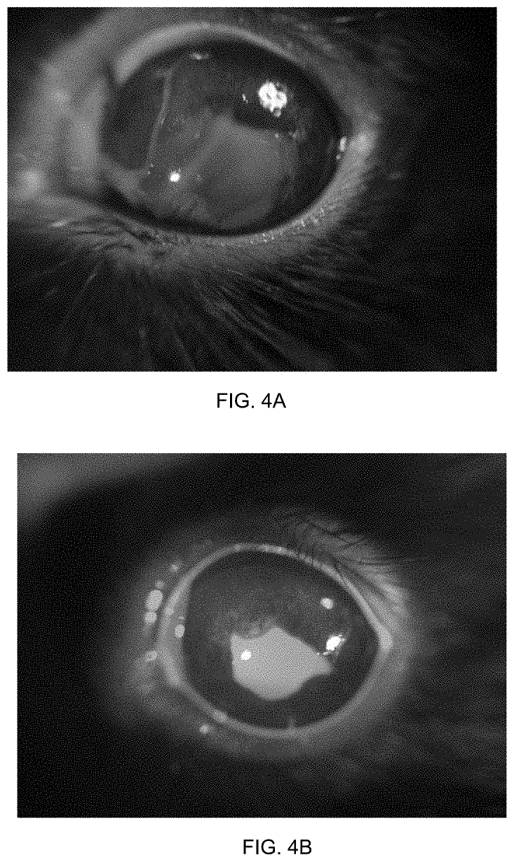

Preliminary results from experiments conducted on Dutch Belted Rabbits are shown in Table 1, below. Table 1 represents the data on keratocytes density from the first group of the animals processed in the experiment. These results agree with those reported herein for ex vivo experiments. The eyes of both experimental groups (Group 2: epithelium debrided, no laser treated and Group 3: epithelium debrided and laser treated) show that 5 hours following surgical removal of the corneal epithelium, the keratocytes in the anterior stroma decrease in number. However, the changes in the keratocyte density of the epithelium debrided and laser treated corneas are not such as large (18-24%) as compared to the debrided but not laser treated corneas (41%). Results shown in FIGS. 4A and 4B further demonstrate that laser treatment of epithelium debrided rabbit eyes notably improves healing relative to non-laser treatment. Similar results are expected in eyes subjected to stromal injury.

TABLE 1

| | [symbol] nsity of stromal keratocytes | | |
|---|---|---|---|
| | calculated per 500,000 [symbol] volume | | |
| | | [symbol] rs after | |
| | [symbol] ial, before | the epithelium | [symbol] anges |
| Animal ID and No. | the epithelium | [symbol] riding (B) | of B/A, |

| | | |
|---|---|---|
| [symbol] ithelium debriding and laser | [symbol] | [symbol] |
| [symbol] atment, | | |
| [symbol] ithelium debriding and laser | [symbol] | [symbol] |
| [symbol] atment, Group 3 | | |
| [symbol] ithelium debrided, no laser | [symbol] | [symbol] |
| treatment, [symbol] up 2 | | |

[symbol]

indicates data missing or illegible when filed

The average changes in the keratocyte density of the epithelium-debrided and laser-treated corneas is 19% comparing to the initial point. These results are shown in Table 2 below. The debrided but not laser treated corneas show keratocyte losses of more than 52%.

TABLE 2

| | | | Cell count per volume (50 μm voxel depth | | | |
|---|---|---|---|---|---|---|
| | | | | 4-5 H POST | | |
| | | | INITIAL | TREATMENT | Change | Average |
| Laser | EPI OFF | 1367-OD | 440 | 358 | 18.64% | 19.37% |
| treated | EPI OFF | 1368-OD | 283 | 214 | 24.38% | |
| | EPI OFF | 1364-OD | 387 | 290 | 25.06% | |
| | EPI OFF | 1365-OD | 333 | 281 | 15.62% | |
| | EPI OFF | 1366-OD | 312 | 271 | 13.14% | |
| Untreated | EPI OFF | 1364-OS | 223 | 94 | 57.85% | 52.85% |
| Control | EPI OFF | 1365-OS | 333 | 159 | 52.25% | |
| | EPI OFF | 1366-OS | 326 | 129 | 60.43% | |
| | EPI OFF | 1369-OD | 421 | 249 | 40.86% | |

These results demonstrate near-infrared-femtosecond-laser-induced alteration of the epithelia-stroma interactions resulting in stabilization of keratocyte network in response to corneal epithelium injury. The reported effect was obtained using the laser irradiation with energy below biological breakdown and may be used for noninvasive research and clinical applications. The molecular nature of that alteration could drive therapeutic strategies for modulation of corneal wound-healing responses.

Femtosecond Laser Initiated Modulations of Binding Affinity Of IL-1β to its Specific Target Molecules

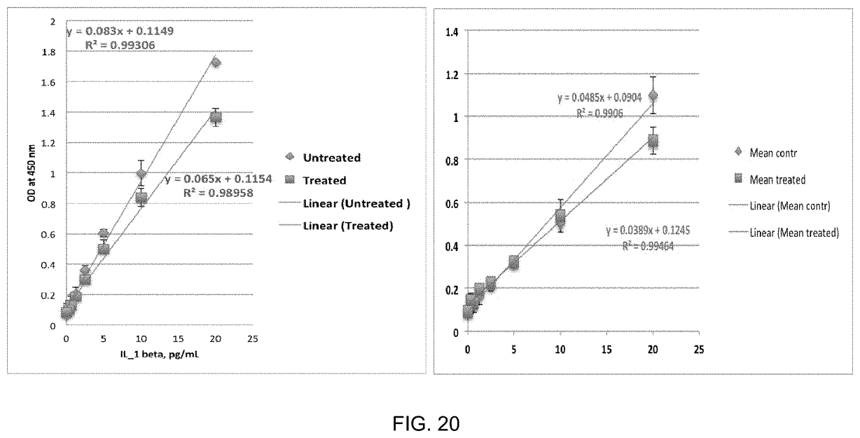

Quantitative ELISA analysis was performed to evaluate the interaction between IL-1β and IL-1β-specific antibodies in an aqueous model. Concentration-signal curves were constructed for the laser-treated and untreated samples of IL-1β solutions, shown in FIGS. 20A and 20B. Results indicate that laser-treated samples (orange squares) have lower angles of inclination (smaller slopes) when compared to the curves of untreated control samples (blue diamonds). These results indicate a decreasing in the binding affinity of the laser treated IL-1β to the specific antibodies. The changes are statistically significant.

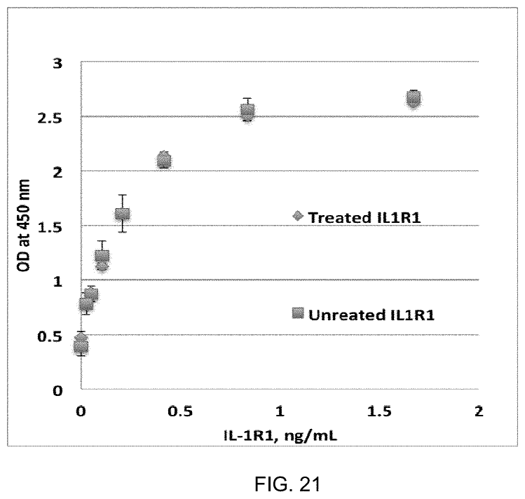

Next, the interaction between IL-1β and IL-1R1 receptors was evaluated using quantitate ELISA analysis. The results, shown in FIG. 21, indicate no significant changes in binding affinity between laser-treated (diamonds) and untreated (squares) IL1R1 samples. Instead, the receptor concentration curves (OD 450 nm) look practically identical for the ELISA of both groups of samples. Without being bound to theory, one possible explanation is that perhaps the changes in aqueous environment caused by femtosecond laser irradiation applied to the IL1R1 solutions in the experiment were not great enough for significant alteration of this ligand-specific antibody interaction.

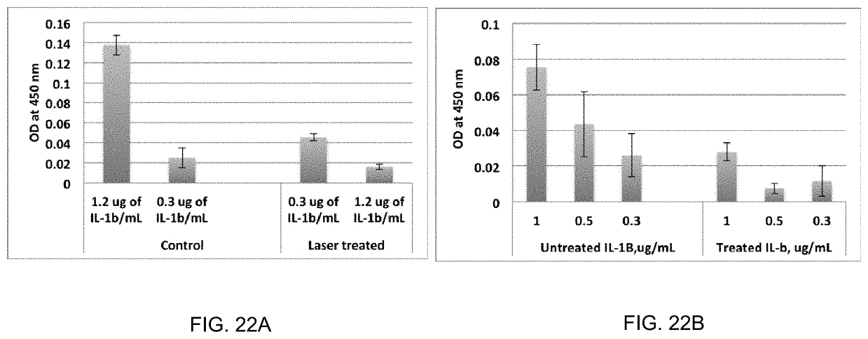

The ligand-receptor interaction ELISA, quantified in FIGS. 22A and 22B, indicates significantly lower binding affinity between the laser-treated IL-1β and its IL1R1 receptor compared to the untreated interleukin samples. The difference between the OD 450 measurement of the laser-treated and control group could reach more than 60%, as determined by ELISA.

### EQUIVALENTS

Although preferred embodiments of the disclosed subject matter have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

What is claimed:

1. A method of modulating cytokine activity within a cornea without treating keratoconus or altering curvature of the cornea, the method comprising:
controlling a light source to apply light energy pulses to corneal tissue;
wherein the light energy pulses:
are below an optical breakdown threshold for the cornea;
ionize water molecules within the treated corneal layer to generate reactive oxygen species; and
modulate cytokine activity.

2. The method of claim 1, wherein the light source is a laser.

3. The method of claim 2, wherein the laser is a femtosecond laser.

4. The method of claim 1, wherein the light energy pulses have an average power output between about 10 mW and about 100 mW.

5. The method of claim 1, wherein the light energy pulses have a pulse energy between about 0.1 nJ and about 10.

6. The method of claim 1, wherein the light energy pulses have a wavelength between about 200 nm and about 1600 nm.

7. The method of claim 1, wherein the light energy pulses have a wavelength that is not significantly absorbed by amino acids in collagen.

8. The method of claim 1, wherein the modulated cytokine activity includes one or more selected from the group consisting of: expression and signaling.

9. The method of claim 8, wherein the expression comprises protein expression.

10. The method of claim 8, wherein the signaling comprises inflammatory signaling.

11. The method of claim 8, wherein the signaling modulates cytokine activity of one or more types of cells.

12. The method of claim 11, wherein the one or more types of cells comprise keratocytes.

13. The method of claim 1, wherein the cytokine is selected from the group consisting of: a chemokine, an interferon, an interleukin, a lymphokine, and a tumor necrosis factor.

14. The method of claim 13 wherein the interleukin includes one or more selected from the group consisting of: interleukin 1 and interleukin 2.

15. The method of claim 1, wherein the method is performed for prophylactic purposes as part of a surgical corrective procedure.

16. The method of claim 15, wherein the surgical corrective procedure is selected from the group consisting of: laser-assisted in situ keratomileusis (LASIK), photorefractive keratectomy (PRK), laser-assisted sub-epithelial keratectomy (LASEK), phakic intraocular lens implantation, radial keratotomy, and cataract surgery.

17. A system for treating a cornea, the system comprising:
a light source configured to project light energy pulses onto at least a portion of a cornea; and
a controller programmed to control the light source in accordance with the method of claim 1.

18. The system of claim 17, further comprising:
an imaging device configured to image the cornea.

19. The system of claim 18, wherein the imaging device is further configured to perform one or more techniques selected from the group consisting of: en face imaging, tomography, and topographic imaging.

20. A system for adapting a laser system for treating a cornea, the system comprising:
laser modification optics adapted and configured to adjust laser output of the laser system; and
a controller programmed to control the laser modification optics as the light source in accordance with the method of claim 1.

21. The system of claim 20, further comprising:
an imaging device configured to image the cornea.

22. The system of claim 21, wherein the imaging device is further configured to perform one or more techniques selected from the group consisting of: en face imaging, tomography, and topographic imaging.

23. A method of preventing or decreasing inflammation, scar formation, or cytokine activity in a non-ophthalmologic and non-arthroscopic procedure, the method comprising:
controlling a light source to apply light energy pulses to non-ophthalmologic and non-cartilaginous tissue;
wherein the light energy pulses:
are below an optical breakdown threshold for the non-ophthalmologic and non-cartilaginous tissue;
ionize water molecules within the treated non-ophthalmologic and non-cartilaginous tissue to generate reactive oxygen species that cross-link collagen within the non-ophthalmologic and non-cartilaginous tissue; and
modulate cytokine activity.

24. The method of claim 23, wherein the non-ophthalmologic and non-cartilaginous tissue is
